(19) United States
(12) Patent Application Publication
(10) Pub. No.: US 2016/0355840 A1
(43) Pub. Date: Dec. 8, 2016

(54) METHODS OF IDENTIFYING AND SELECTING MAIZE PLANTS WITH RESISTANCE TO ANTHRACNOSE STALK ROT

(71) Applicants: E I DU PONT DE NEMOURS AND COMPANY, Wilmington, DE (US); PIONEER HI-BRED INTERNATIONAL INC, Johnston, IA (US)

(72) Inventors: SCOTT B DAVIS, Odessa, DE (US); Jacso Dellai, Passo Fundo (BR); Mark Timothy Jung, West Chester, PA (US); Ana Beatriz Locatelli, Carazinho-RS (BR); Petra J Wolters, Kennett Sqaure, PA (US)

(21) Appl. No.: 15/159,895

(22) Filed: May 20, 2016

Related U.S. Application Data
(60) Provisional application No. 62/170,276, filed on Jun. 3, 2015.

(51) Int. Cl. C12N 15/82; A01H 1/04; C12Q 1/68; A01H 1/02; A01H 5/10

(52) U.S. Cl. CPC C12N 15/8282; A01H 1/02; A01H 5/10; C12Q 1/6895; A01H 1/04; C12Q 2600/13; C12Q 2600/156

(57) ABSTRACT

Compositions and methods useful in identifying and/or selecting maize plants that have anthracnose stalk rot resistance are provided herein. The resistance may be newly conferred or enhanced relative to a control plant. The methods use maize markers on chromosome 10 to identify, select and/or construct resistant plants. Maize plants generated by the methods also provided.

### CROSS REFERENCE TO RELATED APPLICATIONS

This application claims the benefit of U.S. Provisional Application No. 62/170,276, filed Jun. 3, 2015, the entire contents of which are herein incorporated by reference.

### FIELD

The field is related to plant breeding and methods of identifying and selecting plants with resistance to Anthracnose stalk rot.

### REFERENCE TO SEQUENCE LISTING SUBMITTED ELECTRONICALLY

The official copy of the sequence listing is submitted electronically via EFS-Web as an ASCII formatted sequence listing with a file named 20160519_BB2531USNP_SequenceListing_ST25 created on May 19, 2016, has a size of 6 kilobytes, and is filed concurrently with the specification. The sequence listing contained in this ASCII formatted document is part of the specification and is herein incorporated by reference in its entirety.

### BACKGROUND

Anthracnose stalk rot (ASR) caused by the fungal pathogen Colletotrichum graminicola(Ces.) Wils, (Cg) is one of the major stalk rot diseases in maize (Zea mays L.). ASR is a major concern due to significant reduction in yield, grain weight and quality. Yield losses occur from premature plant death that interrupts filling of the grain and from stalk breakage and lodging that causes ears to be lost in the field. ASR occurs in all corn growing areas and can result in 10 to 20% losses. Farmers can combat infection by fungi such as anthracnose through the use of fungicides, but these have environmental side effects and require monitoring of fields and diagnostic techniques to determine which fungus is causing the infection so that the correct fungicide can be used. The use of corn lines that carry genetic or transgenic sources of resistance is more practical if the genes responsible for resistance can be incorporated into elite, high yielding germplasm without reducing yield. Genetic sources of resistance to Cg have been described (White, et al. (1979) Annu. Corn Sorghum Res. Conf. Proc. 34:1-15; Carson. 1981. Sources of inheritance of resistance to anthracnose stalk rot of corn. Ph.D. Thesis, University of Illinois, Urbana-Champaign; Badu-Apraku et al., (1987) Phytopathology 77:957-959; Toman et al. 1993. Phytopathology, 83:981-986; Cowen, N et al. (1991) Maize Genetics Conference Abstracts 33; Jung, et al., 1994. Theoretical and Applied Genetics, 89:413-418). However, introgression of resistance can be highly complex.

Selection through the use of molecular markers associated with the anthracnose stalk rot resistance trait allows selections based solely on the genetic composition of the progeny. As a result, plant breeding can occur more rapidly, thereby generating commercially acceptable maize plants with a higher level of anthracnose stalk rot. There are multiple QTL controlling resistance to anthracnose stalk rot (e.g. rcg1 and rcg1b on chromosome 4 (WO2008157432 and WO2006107931)), with each having a different effect on the trait. Thus, it is desirable to provide compositions and methods for identifying and selecting maize plants with newly conferred or enhanced anthracnose stalk rot resistance. These plants can be used in breeding programs to generate high-yielding hybrids that are resistant to anthracnose stalk rot.

### SUMMARY

Compositions and methods useful in identifying and selecting maize plants with anthracnose stalk rot resistance are provided herein. The methods use markers to identify and/or select resistant plants or to identify and/or counter-select susceptible plants. Maize plants having newly conferred or enhanced resistance to anthracnose stalk rot relative to control plants are also provided herein.

In one embodiment, methods for identifying and/or selecting maize plants having resistance to anthracnose stalk rot are presented. In these methods DNA of a maize plant is analyzed for the presence of a QTL allele on chromosome 10 that is associated with anthracnose stalk rot resistance, wherein said QTL comprises: a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35; and a maize plant is identified and/or selected as having anthracnose stalk rot resistance if said QTL allele is detected. The selected maize plant may be crossed to a second maize plant in order to obtain a progeny plant that has the QTL allele. The anthracnose stalk rot resistance may be newly conferred or enhanced relative to a control plant that does not have the favorable QTL allele. The QTL allele may be further refined to a chromosomal interval defined by and including markers C00429-801 and PHM824 or still further a chromosomal interval defined by and including markers SYN17244 and sbd_INBREDA_48 or still further a chromosomal interval defined by and including markers sbd_INBREDA_093 and sbd_INBREDA_109. The analyzing step may be performed by isolating nucleic acids and detecting one or more marker alleles linked to and associated with the QTL allele.

In another embodiment, methods of identifying and/or selecting maize plants with anthracnose stalk rot resistance are provided in which one or more marker alleles linked to and associated with any of: a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35, are detected in a maize plant, and a maize plant having the one or more marker alleles is selected. The one or more marker alleles may be linked by 10 cM, 9 cM, 8 cM, 7 cM, 6 cM, 5 cM, 4 cM, 3 cM, 2 cM, 1 cM, 0.9 cM, 0.8 cM, 0.7 cM, 0.6 cM, 0.5 cM, 0.4 cM, 0.3 cM, 0.2 cM, or 0.1 cM or less on a single meiosis based genetic map. The selected maize plant may be crossed to a second maize plant to obtain a progeny plant that has one or more marker alleles linked to and associated with any of: a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35.

In another embodiment, methods of identifying and/or selecting maize plants with anthracnose stalk rot resistance are provided in which one or more marker alleles linked to and associated with a haplotype comprising: a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35, are detected in a maize plant, and a maize plant having the one or more marker alleles is selected. The one or more marker alleles may be linked to the haplotype by 10 cM, 9 cM, 8 cM, 7 cM, 6 cM, 5 cM, 4 cM, 3 cM, 2 cM, 1 cM, 0.9 cM, 0.8 cM, 0.7 cM, 0.6 cM, 0.5 cM, 0.4 cM, 0.3 cM, 0.2 cM, or 0.1 cM or less on a single meiosis based genetic map. The selected maize plant may be crossed to a second maize plant to obtain a progeny plant that has one or more marker alleles linked to and associated with a haplotype comprising: a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35.

In another embodiment, methods of identifying and/or selecting maize plants with anthracnose stalk rot resistance are provided in which a haplotype comprising: a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35; is detected in a maize plant, and a maize plant having the one or more marker alleles is selected. A selected maize plant may be crossed to a second maize plant to obtain a progeny plant that has the haplotype comprising: a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35.

In another embodiment, methods of introgressing a QTL allele associated with anthracnose stalk rot resistance are presented herein. In these methods, a population of maize plants is screened with one or more markers to determine if any of the maize plants has a QTL allele associated with anthracnose stalk rot resistance, and at least one maize plant that has the QTL allele associated with anthracnose stalk rot resistance is selected from the population. The QTL allele comprises a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35. The one or more markers used for screening can be located within 5 cM, 2 cM, or 1 cM (on a single meiosis based genetic map) of any of a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, and a “G” at sbd_INBREDA_35.

Maize plants identified and/or selected using any of the methods presented above are also provided.

### BRIEF DESCRIPTION OF THE SEQUENCE LISTING

The disclosure can be more fully understood from the following detailed description and the Sequence Listing which forms a part of this application.

The sequence descriptions and Sequence Listing attached hereto comply with the rules governing nucleotide and/or amino acid sequence disclosures in patent applications as set forth in 37 C.F.R. §1.821 1.825. The Sequence Listing contains the one letter code for nucleotide sequence characters and the three letter codes for amino acids as defined in conformity with the IUPAC IUBMB standards described in Nucleic Acids Res. 13:3021 3030 (1985) and in the Biochemical J. 219 (2):345 373 (1984) which are herein incorporated by reference. The symbols and format used for nucleotide and amino acid sequence data comply with the rules set forth in 37 C.F.R. §1.822.

SEQ ID NO:1 is the reference sequence for marker C00429-801.

SEQ ID NO:2 is the reference sequence for marker SYN17615.

SEQ ID NO:3 is the reference sequence for marker PZE-110006361.

SEQ ID NO:4 is the reference sequence for marker PHM824-17.

SEQ ID NO:5 is the reference sequence for marker SYN17244.

SEQ ID NO:6 is the reference sequence for marker sbd_INBREDA_4.

SEQ ID NO:7 is the reference sequence for marker sbd_INBREDA_9.

SEQ ID NO:8 is the reference sequence for marker sbd_INBREDA_13.

SEQ ID NO:9 is the reference sequence for marker sbd_INBREDA_24.

SEQ ID NO:10 is the reference sequence for marker sbd_INBREDA_25.

SEQ ID NO:11 is the reference sequence for marker sbd_INBREDA_32.

SEQ ID NO:12 is the reference sequence for marker sbd_INBREDA_33.

SEQ ID NO:13 is the reference sequence for marker sbd_INBREDA_35.

SEQ ID NO:14 is the reference sequence for marker sbd_INBREDA_48.

SEQ ID NO:15 is the reference sequence for marker sbd_INBREDA_093.

SEQ ID NO:16 is the reference sequence for marker sbd_INBREDA_109.

### DETAILED DESCRIPTION

Maize marker loci that demonstrate statistically significant co-segregation with the anthracnose stalk rot resistance trait are provided herein. Detection of these loci or additional linked loci can be used in marker assisted selection as part of a maize breeding program to produce maize plants that have resistance to anthracnose stalk rot.

The following definitions are provided as an aid to understand the present disclosure.

It is to be understood that the disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a”, “an” and “the”, for example, include plural referents unless the content clearly dictates otherwise. Thus, for example, reference to “plant”, “the plant” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Unless otherwise indicated, nucleic acids are written left to right in 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the disclosure pertains. Although any methods and materials similar or equivalent to those described herein can be used for testing of the subject matter recited in the current disclosure, the preferred materials and methods are described herein. In describing and claiming the subject matter of the current disclosure, the following terminology will be used in accordance with the definitions set out below.

The term “allele” refers to one of two or more different nucleotide sequences that occur at a specific locus.

“Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An “amplicon” is an amplified nucleic acid, e.g., a nucleic acid that is produced by amplifying a template nucleic acid by any available amplification method (e.g., PCR, LCR, transcription, or the like).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

The term “assemble” applies to BACs and their propensities for coming together to form contiguous stretches of DNA. A BAC “assembles” to a contig based on sequence alignment, if the BAC is sequenced, or via the alignment of its BAC fingerprint to the fingerprints of other BACs. Public assemblies can be found using the Maize Genome Browser, which is publicly available on the internet.

An allele is “associated with” a trait when it is part of or linked to a DNA sequence or allele that affects the expression of the trait. The presence of the allele is an indicator of how the trait will be expressed.

A “BAC”, or bacterial artificial chromosome, is a cloning vector derived from the naturally occurring F factor of Escherichia coli, which itself is a DNA element that can exist as a circular plasmid or can be integrated into the bacterial chromosome. BACs can accept large inserts of DNA sequence. In maize, a number of BACs each containing a large insert of maize genomic DNA from maize inbred line B73, have been assembled into contigs (overlapping contiguous genetic fragments, or “contiguous DNA”), and this assembly is available publicly on the internet.

A BAC fingerprint is a means of analyzing similarity between several DNA samples based upon the presence or absence of specific restriction sites (restriction sites being nucleotide sequences recognized by enzymes that cut or “restrict” the DNA). Two or more BAC samples are digested with the same set of restriction enzymes and the sizes of the fragments formed are compared, usually using gel separation.

“Backcrossing” refers to the process whereby hybrid progeny are repeatedly crossed back to one of the parents. In a backcrossing scheme, the “donor” parent refers to the parental plant with the desired gene/genes, locus/loci, or specific phenotype to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F₁ generation; the term “BC₁” then refers to the second use of the recurrent parent, “BC₂” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

A “chromosome” is a single piece of coiled DNA containing many genes that act and move as a unity during cell division and therefore can be said to be linked. It can also be referred to as a “linkage group”.

The phrase “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to anthracnose stalk rot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

The term “complement” refers to a nucleotide sequence that is complementary to a given nucleotide sequence, i.e. the sequences are related by the Watson-Crick base-pairing rules.

The term “contiguous DNA” refers to an uninterrupted stretch of genomic DNA represented by partially overlapping pieces or contigs.

When referring to the relationship between two genetic elements, such as a genetic element contributing to anthracnose stalk rot resistance and a proximal marker, “coupling” phase linkage indicates the state where the “favorable” allele at the anthracnose stalk rot resistance locus is physically associated on the same chromosome strand as the “favorable” allele of the respective linked marker locus. In coupling phase, both favorable alleles are inherited together by progeny that inherit that chromosome strand.

The term “crossed” or “cross” refers to a sexual cross and involved the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

A plant referred to herein as “diploid” has two sets (genomes) of chromosomes.

A plant referred to herein as a “doubled haploid” is developed by doubling the haploid set of chromosomes (i.e., half the normal number of chromosomes). A doubled haploid plant has two identical sets of chromosomes, and all loci are considered homozygous.

An “elite line” is any line that has resulted from breeding and selection for superior agronomic performance.

An “exotic maize strain” or an “exotic maize germplasm” is a strain derived from a maize plant not belonging to an available elite maize line or strain of germplasm. In the context of a cross between two maize plants or strains of germplasm, an exotic germplasm is not closely related by descent to the elite germplasm with which it is crossed. Most commonly, the exotic germplasm is not derived from any known elite line of maize, but rather is selected to introduce novel genetic elements (typically novel alleles) into a breeding program.

A “favorable allele” is the allele at a particular locus (a marker, a QTL, etc.) that confers, or contributes to, an agronomically desirable phenotype, e.g., anthracnose stalk rot resistance, and that allows the identification of plants with that agronomically desirable phenotype. A favorable allele of a marker is a marker allele that segregates with the favorable phenotype.

“Fragment” is intended to mean a portion of a nucleotide sequence. Fragments can be used as hybridization probes or PCR primers using methods disclosed herein.

A “genetic map” is a description of genetic linkage relationships among loci on one or more chromosomes (or linkage groups) within a given species, generally depicted in a diagrammatic or tabular form. For each genetic map, distances between loci are measured by how frequently their alleles appear together in a population (their recombination frequencies). Alleles can be detected using DNA or protein markers, or observable phenotypes. A genetic map is a product of the mapping population, types of markers used, and the polymorphic potential of each marker between different populations. Genetic distances between loci can differ from one genetic map to another. However, information can be correlated from one map to another using common markers. One of ordinary skill in the art can use common marker positions to identify positions of markers and other loci of interest on each individual genetic map. The order of loci should not change between maps, although frequently there are small changes in marker orders due to e.g. markers detecting alternate duplicate loci in different populations, differences in statistical approaches used to order the markers, novel mutation or laboratory error.

A “genetic map location” is a location on a genetic map relative to surrounding genetic markers on the same linkage group where a specified marker can be found within a given species.

“Genetic mapping” is the process of defining the linkage relationships of loci through the use of genetic markers, populations segregating for the markers, and standard genetic principles of recombination frequency.

“Genetic markers” are nucleic acids that are polymorphic in a population and where the alleles of which can be detected and distinguished by one or more analytic methods, e.g., RFLP, AFLP, isozyme, SNP, SSR, and the like. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

“Genetic recombination frequency” is the frequency of a crossing over event (recombination) between two genetic loci. Recombination frequency can be observed by following the segregation of markers and/or traits following meiosis.

“Genome” refers to the total DNA, or the entire set of genes, carried by a chromosome or chromosome set.

The term “genotype” is the genetic constitution of an individual (or group of individuals) at one or more genetic loci. Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture, or more generally, all individuals within a species or for several species (e.g., maize germplasm collection or Andean germplasm collection). The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, that can be cultured into a whole plant.

A plant referred to as “haploid” has a single set (genome) of chromosomes.

A “haplotype” is the genotype of an individual at a plurality of genetic loci, i.e. a combination of alleles. Typically, the genetic loci described by a haplotype are physically and genetically linked, i.e., on the same chromosome segment.

The term “heterogeneity” is used to indicate that individuals within the group differ in genotype at one or more specific loci.

The heterotic response of material, or “heterosis”, can be defined by performance which exceeds the average of the parents (or high parent) when crossed to other dissimilar or unrelated groups.

A “heterotic group” comprises a set of genotypes that perform well when crossed with genotypes from a different heterotic group (Hallauer et al. (1998) Corn breeding, p. 463-564. In G. F. Sprague and J. W. Dudley (ed.) Corn and corn improvement). Inbred lines are classified into heterotic groups, and are further subdivided into families within a heterotic group, based on several criteria such as pedigree, molecular marker-based associations, and performance in hybrid combinations (Smith et al. (1990) Theor. Appl. Gen. 80:833-840). The two most widely used heterotic groups in the United States are referred to as “Iowa Stiff Stalk Synthetic” (also referred to herein as “stiff stalk”) and “Lancaster” or “Lancaster Sure Crop” (sometimes referred to as NSS, or non-Stiff Stalk).

Some heterotic groups possess the traits needed to be a female parent, and others, traits for a male parent. For example, in maize, yield results from public inbreds released from a population called BSSS (Iowa Stiff Stalk Synthetic population) has resulted in these inbreds and their derivatives becoming the female pool in the central Corn Belt. BSSS inbreds have been crossed with other inbreds, e.g. SD 105 and Maiz Amargo, and this general group of materials has become known as Stiff Stalk Synthetics (SSS) even though not all of the inbreds are derived from the original BSSS population (Mikel and Dudley (2006) Crop Sci: 46:1193-1205). By default, all other inbreds that combine well with the SSS inbreds have been assigned to the male pool, which for lack of a better name has been designated as NSS, i.e. Non-Stiff Stalk. This group includes several major heterotic groups such as Lancaster Surecrop, Iodent, and Leaming Corn.

An individual is “heterozygous” if more than one allele type is present at a given locus (e.g., a diploid individual with one copy each of two different alleles).

The term “homogeneity” indicates that members of a group have the same genotype at one or more specific loci.

An individual is “homozygous” if the individual has only one type of allele at a given locus (e.g., a diploid individual has a copy of the same allele at a locus for each of two homologous chromosomes).

The term “hybrid” refers to the progeny obtained between the crossing of at least two genetically dissimilar parents.

“Hybridization” or “nucleic acid hybridization” refers to the pairing of complementary RNA and DNA strands as well as the pairing of complementary DNA single strands.

The term “hybridize” means to form base pairs between complementary regions of nucleic acid strands.

An “IBM genetic map” can refer to any of following maps: IBM, IBM2, IBM2 neighbors, IBM2 FPC0507, IBM2 2004 neighbors, IBM2 2005 neighbors, IBM2 2005 neighbors frame, IBM2 2008 neighbors, IBM2 2008 neighbors frame, or the latest version on the maizeGDB website. IBM genetic maps are based on a B73×Mo17 population in which the progeny from the initial cross were random-mated for multiple generations prior to constructing recombinant inbred lines for mapping. Newer versions reflect the addition of genetic and BAC mapped loci as well as enhanced map refinement due to the incorporation of information obtained from other genetic maps or physical maps, cleaned date, or the use of new algorithms.

The term “inbred” refers to a line that has been bred for genetic homogeneity.

The term “indel” refers to an insertion or deletion, wherein one line may be referred to as having an inserted nucleotide or piece of DNA relative to a second line, or the second line may be referred to as having a deleted nucleotide or piece of DNA relative to the first line.

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background.

The process of “introgressing” is often referred to as “backcrossing” when the process is repeated two or more times.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor.

As used herein, the term “linkage” is used to describe the degree with which one marker locus is associated with another marker locus or some other locus. The linkage relationship between a molecular marker and a locus affecting a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units (or cM) of a single meiosis map (a genetic map based on a population that has undergone one round of meiosis, such as e.g. an F₂; the IBM2 maps consist of multiple meioses). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “in proximity to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

Linkage disequilibrium is most commonly assessed using the measure r², which is calculated using the formula described by Hill, W. G. and Robertson, A, Theor. Appl. Genet. 38:226-231(1968). When r²=1, complete LD exists between the two marker loci, meaning that the markers have not been separated by recombination and have the same allele frequency. The r² value will be dependent on the population used. Values for r² above ⅓ indicate sufficiently strong LD to be useful for mapping (Ardlie et al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

A “locus” is a position on a chromosome, e.g. where a nucleotide, gene, sequence, or marker is located.

The “logarithm of odds (LOD) value” or “LOD score” (Risch, Science 255:803-804 (1992)) is used in genetic interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. LOD scores can also be used to show the strength of association between marker loci and quantitative traits in “quantitative trait loci” mapping. In this case, the LOD score's size is dependent on the closeness of the marker locus to the locus affecting the quantitative trait, as well as the size of the quantitative trait effect.

“Maize” refers to a plant of the Zea mays L. ssp. mays and is also known as “corn”.

The term “maize plant” includes whole maize plants, maize plant cells, maize plant protoplast, maize plant cell or maize tissue culture from which maize plants can be regenerated, maize plant calli, maize plant clumps and maize plant cells that are intact in maize plants or parts of maize plants, such as maize seeds, maize cobs, maize flowers, maize cotyledons, maize leaves, maize stems, maize buds, maize roots, maize root tips and the like.

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker will consist of complementary primers flanking the locus and/or complementary probes that hybridize to polymorphic alleles at the locus. A DNA marker, or a genetic marker, can also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus is the locus (gene, sequence or nucleotide) that the marker detects.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population.

“Marker assisted selection” (of MAS) is a process by which individual plants are selected based on marker genotypes.

“Marker assisted counter-selection” is a process by which marker genotypes are used to identify plants that will not be selected, allowing them to be removed from a breeding program or planting.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

An allele “negatively” correlates with a trait when it is linked to it and when presence of the allele is an indicator that a desired trait or trait form will not occur in a plant comprising the allele.

“Nucleotide sequence”, “polynucleotide”, “nucleic acid sequence”, and “nucleic acid fragment” are used interchangeably and refer to a polymer of RNA or DNA that is single- or double-stranded, optionally containing synthetic, non-natural or altered nucleotide bases. A “nucleotide” is a monomeric unit from which DNA or RNA polymers are constructed, and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “phenotype”, “phenotypic trait”, or “trait” can refer to the observable expression of a gene or series of genes. The phenotype can be observable to the naked eye, or by any other means of evaluation known in the art, e.g., weighing, counting, measuring (length, width, angles, etc.), microscopy, biochemical analysis, or an electromechanical assay. In some cases, a phenotype is directly controlled by a single gene or genetic locus, i.e., a “single gene trait” or a “simply inherited trait”. In the absence of large levels of environmental variation, single gene traits can segregate in a population to give a “qualitative” or “discrete” distribution, i.e. the phenotype falls into discrete classes. In other cases, a phenotype is the result of several genes and can be considered a “multigenic trait” or a “complex trait”. Multigenic traits segregate in a population to give a “quantitative” or “continuous” distribution, i.e. the phenotype cannot be separated into discrete classes. Both single gene and multigenic traits can be affected by the environment in which they are being expressed, but multigenic traits tend to have a larger environmental component.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A maize plant “derived from an inbred in the Stiff Stalk Synthetic population” may be a hybrid.

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”.

An allele “positively” correlates with a trait when it is linked to it and when presence of the allele is an indicator that the desired trait or trait form will occur in a plant comprising the allele.

The “probability value” or “p-value” is the statistical likelihood that the particular combination of a phenotype and the presence or absence of a particular marker allele is random. Thus, the lower the probability score, the greater the likelihood that a locus and a phenotype are associated. The probability score can be affected by the proximity of the first locus (usually a marker locus) and the locus affecting the phenotype, plus the magnitude of the phenotypic effect (the change in phenotype caused by an allele substitution). In some aspects, the probability score is considered “significant” or “nonsignificant”. In some embodiments, a probability score of 0.05 (p=0.05, or a 5% probability) of random assortment is considered a significant indication of association. However, an acceptable probability can be any probability of less than 50% (p=0.5). For example, a significant probability can be less than 0.25, less than 0.20, less than 0.15, less than 0.1, less than 0.05, less than 0.01, or less than 0.001.

A “production marker” or “production SNP marker” is a marker that has been developed for high-throughput purposes. Production SNP markers are developed to detect specific polymorphisms and are designed for use with a variety of chemistries and platforms. The marker names used here begin with a PHM prefix to denote ‘Pioneer Hi-Bred Marker’, followed by a number that is specific to the sequence from which it was designed, followed by a “.” or a “-” and then a suffix that is specific to the DNA polymorphism. A marker version can also follow (A, B, C etc.) that denotes the version of the marker designed to that specific polymorphism.

The term “progeny” refers to the offspring generated from a cross.

A “progeny plant” is a plant generated from a cross between two plants.

The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question.

A “reference sequence” or a “consensus sequence” is a defined sequence used as a basis for sequence comparison. The reference sequence for a PHM marker is obtained by sequencing a number of lines at the locus, aligning the nucleotide sequences in a sequence alignment program (e.g. Sequencher), and then obtaining the most common nucleotide sequence of the alignment. Polymorphisms found among the individual sequences are annotated within the consensus sequence. A reference sequence is not usually an exact copy of any individual DNA sequence, but represents an amalgam of available sequences and is useful for designing primers and probes to polymorphisms within the sequence.

In “repulsion” phase linkage, the “favorable” allele at the locus of interest is physically linked with an “unfavorable” allele at the proximal marker locus, and the two “favorable” alleles are not inherited together (i.e., the two loci are “out of phase” with each other).

As used herein, “anthracnose stalk rot resistance” refers to enhanced resistance or tolerance to a fungal pathogen that causes anthracnose stalk rot when compared to a control plant. Effects may vary from a slight increase in tolerance to the effects of the fungal pathogen (e.g., partial inhibition) to total resistance such that the plant is unaffected by the presence of the fungal pathogen. An increased level of resistance against a particular fungal pathogen or against a wider spectrum of fungal pathogens constitutes “enhanced” or improved fungal resistance. The embodiments of the disclosure will enhance or improve resistance to the fungal pathogen that causes anthracnose stalk rot, such that the resistance of the plant to a fungal pathogen or pathogens will increase. The term “enhance” refers to improve, increase, amplify, multiply, elevate, raise, and the like. Thus, plants described herein as being resistant to anthracnose stalk rot can also be described as being resistant to infection by Colletotrichum graminicola or having ‘enhanced resistance’ to infection by Colletotrichum graminicola.

A “topcross test” is a test performed by crossing each individual (e.g. a selection, inbred line, clone or progeny individual) with the same pollen parent or “tester”, usually a homozygous line.

The phrase “under stringent conditions” refers to conditions under which a probe or polynucleotide will hybridize to a specific nucleic acid sequence, typically in a complex mixture of nucleic acids, but to essentially no other sequences. Stringent conditions are sequence-dependent and will be different in different circumstances. Longer sequences hybridize specifically at higher temperatures. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3, and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C., depending on primer length. Additional guidelines for determining hybridization parameters are provided in numerous references.

An “unfavorable allele” of a marker is a marker allele that segregates with the unfavorable plant phenotype, therefore providing the benefit of identifying plants that can be removed from a breeding program or planting.

The term “yield” refers to the productivity per unit area of a particular plant product of commercial value. For example, yield of maize is commonly measured in bushels of seed per acre or metric tons of seed per hectare per season. Yield is affected by both genetic and environmental factors. “Agronomics”, “agronomic traits”, and “agronomic performance” refer to the traits (and underlying genetic elements) of a given plant variety that contribute to yield over the course of growing season. Individual agronomic traits include emergence vigor, vegetative vigor, stress tolerance, disease resistance or tolerance, herbicide resistance, branching, flowering, seed set, seed size, seed density, standability, threshability and the like. Yield is, therefore, the final culmination of all agronomic traits.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the CLUSTAL V method of alignment (Higgins and Sharp, CABIOS. 5:151 153 (1989)) with the default parameters (GAP PENALTY=10, GAP LENGTH PENALTY=10). Default parameters for pairwise alignments and calculation of percent identity of protein sequences using the CLUSTAL V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the CLUSTAL V program, it is possible to obtain “percent identity” and “divergence” values by viewing the “sequence distances” table on the same program; unless stated otherwise, percent identities and divergences provided and claimed herein were calculated in this manner.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

### Genetic Mapping

It has been recognized for quite some time that specific genetic loci correlating with particular phenotypes, such as resistance to anthracnose stalk rot, can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS).

A variety of methods well known in the art are available for detecting molecular markers or clusters of molecular markers that co-segregate with a trait of interest, such as the anthracnose stalk rot resistance trait. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference. Two such methods used to detect trait loci of interest are: 1) Population-based association analysis (i.e. association mapping) and 2) Traditional linkage analysis.

### Association Mapping

Understanding the extent and patterns of linkage disequilibrium (LD) in the genome is a prerequisite for developing efficient association approaches to identify and map quantitative trait loci (QTL). Linkage disequilibrium (LD) refers to the non-random association of alleles in a collection of individuals. When LD is observed among alleles at linked loci, it is measured as LD decay across a specific region of a chromosome. The extent of the LD is a reflection of the recombinational history of that region. The average rate of LD decay in a genome can help predict the number and density of markers that are required to undertake a genome-wide association study and provides an estimate of the resolution that can be expected.

Association or LD mapping aims to identify significant genotype-phenotype associations. It has been exploited as a powerful tool for fine mapping in outcrossing species such as humans (Corder et al. (1994) “Protective effect of apolipoprotein-E type-2 allele for late-onset Alzheimer-disease,” Nat Genet 7:180-184; Hastbacka et al. (1992) “Linkage disequilibrium mapping in isolated founder populations: diastrophic dysplasia in Finland,” Nat Genet 2:204-211; Kerem et al. (1989) “Identification of the cystic fibrosis gene: genetic analysis,” Science 245:1073-1080) and maize (Remington et al., (2001) “Structure of linkage disequilibrium and phenotype associations in the maize genome,” Proc Natl Acad Sci USA 98:11479-11484; Thornsberry et al. (2001) “Dwarf8 polymorphisms associate with variation in flowering time,” Nat Genet 28:286-289; reviewed by Flint-Garcia et al. (2003) “Structure of linkage disequilibrium in plants,” Annu Rev Plant Biol. 54:357-374), where recombination among heterozygotes is frequent and results in a rapid decay of LD. In inbreeding species where recombination among homozygous genotypes is not genetically detectable, the extent of LD is greater (i.e., larger blocks of linked markers are inherited together) and this dramatically enhances the detection power of association mapping (Wall and Pritchard (2003) “Haplotype blocks and linkage disequilibrium in the human genome,” Nat Rev Genet 4:587-597).

The recombinational and mutational history of a population is a function of the mating habit as well as the effective size and age of a population. Large population sizes offer enhanced possibilities for detecting recombination, while older populations are generally associated with higher levels of polymorphism, both of which contribute to observably accelerated rates of LD decay. On the other hand, smaller effective population sizes, e.g., those that have experienced a recent genetic bottleneck, tend to show a slower rate of LD decay, resulting in more extensive haplotype conservation (Flint-Garcia et al. (2003) “Structure of linkage disequilibrium in plants,” Annu Rev Plant Biol. 54:357-374).

Elite breeding lines provide a valuable starting point for association analyses. Association analyses use quantitative phenotypic scores (e.g., disease tolerance rated from one to nine for each maize line) in the analysis (as opposed to looking only at tolerant versus resistant allele frequency distributions in intergroup allele distribution types of analysis). The availability of detailed phenotypic performance data collected by breeding programs over multiple years and environments for a large number of elite lines provides a valuable dataset for genetic marker association mapping analyses. This paves the way for a seamless integration between research and application and takes advantage of historically accumulated data sets. However, an understanding of the relationship between polymorphism and recombination is useful in developing appropriate strategies for efficiently extracting maximum information from these resources.

This type of association analysis neither generates nor requires any map data, but rather is independent of map position. This analysis compares the plants' phenotypic score with the genotypes at the various loci. Subsequently, any suitable maize map (for example, a composite map) can optionally be used to help observe distribution of the identified QTL markers and/or QTL marker clustering using previously determined map locations of the markers.

### Traditional Linkage Analysis

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

Maize marker loci that demonstrate statistically significant co-segregation with the anthracnose stalk rot resistance trait, as determined by traditional linkage analysis and by whole genome association analysis, are provided herein. Detection of these loci or additional linked loci can be used in marker assisted maize breeding programs to produce plants having resistance to anthracnose stalk rot.

Activities in marker assisted maize breeding programs may include but are not limited to: selecting among new breeding populations to identify which population has the highest frequency of favorable nucleic acid sequences based on historical genotype and agronomic trait associations, selecting favorable nucleic acid sequences among progeny in breeding populations, selecting among parental lines based on prediction of progeny performance, and advancing lines in germplasm improvement activities based on presence of favorable nucleic acid sequences.

### QTL Locations

A QTL on chromosome 10 was identified as being associated with the anthracnose stalk rot resistance trait using traditional linkage mapping (Example 1). The QTL is located on chromosome 10 in a region defined by and including C00429-801 and PHM824, a subinterval of which is defined by and includes SYN17244 and sbd_INBREDA_48, a subinterval of which is defined by and includes markers sbd_INBREDA_093 and sbd_INBREDA_109.

### Chromosomal Intervals

Chromosomal intervals that correlate with the anthracnose stalk rot resistance trait are provided. A variety of methods well known in the art are available for identifying chromosomal intervals. The boundaries of such chromosomal intervals are drawn to encompass markers that will be linked to the gene(s) controlling the trait of interest. In other words, the chromosomal interval is drawn such that any marker that lies within that interval (including the terminal markers that define the boundaries of the interval) can be used as a marker for the anthracnose stalk rot resistance trait. Tables 1 and 2 identify markers within the chromosome 10 QTL region that were shown herein to associate with the anthracnose stalk rot resistance trait and that are linked to a gene(s) controlling anthracnose stalk rot resistance. Reference sequences for each of the markers are represented by SEQ ID NOs:1-16.

Each interval comprises at least one QTL, and furthermore, may indeed comprise more than one QTL. Close proximity of multiple QTL in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identify the same QTL or two different QTL. Regardless, knowledge of how many QTL are in a particular interval is not necessary to make or practice that which is presented in the current disclosure.

The chromosome 10 interval may encompass any of the markers identified herein as being associated with the anthracnose stalk rot resistance trait including: C00429-801, SYN17615, PZE-110006361, PHM824-17, SYN17244, sbd_INBREDA_4, sbd_INBREDA_9, sbd_INBREDA_13, sbd_INBREDA_24, sbd_INBREDA_25, sbd_INBREDA_32, sbd_INBREDA_33, sbd_INBREDA_35, sbd_INBREDA_48, sbd_INBREDA_093, and sbd_INBREDA_109. The chromosome 10 interval, for example, may be defined by markers C00429-801 and PHM824-17, a further subinterval of which can be defined by markers SYN17244 and sbd_INBREDA_48, a further subinterval of which can be defined by markers sbd_INBREDA_093 and sbd_INBREDA_109. Any marker located within these intervals can find use as a marker for anthracnose stalk rot resistance and can be used in the context of the methods presented herein to identify and/or select maize plants that have resistance to anthracnose stalk rot, whether it is newly conferred or enhanced compared to a control plant.

Chromosomal intervals can also be defined by markers that are linked to (show linkage disequilibrium with) a QTL marker, and r² is a common measure of linkage disequilibrium (LD) in the context of association studies. If the r² value of LD between a chromosome 10 marker locus in an interval of interest and another chromosome 10 marker locus in close proximity is greater than ⅓ (Ardlie et al., Nature Reviews Genetics 3:299-309 (2002)), the loci are in linkage disequilibrium with one another.

### Markers and Linkage Relationships

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are said to be “proximal to” each other.

Although particular marker alleles can co-segregate with the anthracnose stalk rot resistance trait, it is important to note that the marker locus is not necessarily responsible for the expression of the anthracnose stalk rot resistant phenotype. For example, it is not a requirement that the marker polynucleotide sequence be part of a gene that is responsible for the anthracnose stalk rot resistant phenotype (for example, is part of the gene open reading frame). The association between a specific marker allele and the anthracnose stalk rot resistance trait is due to the original “coupling” linkage phase between the marker allele and the allele in the ancestral maize line from which the allele originated. Eventually, with repeated recombination, crossing over events between the marker and genetic locus can change this orientation. For this reason, the favorable marker allele may change depending on the linkage phase that exists within the parent having resistance to anthracnose stalk rot that is used to create segregating populations. This does not change the fact that the marker can be used to monitor segregation of the phenotype. It only changes which marker allele is considered favorable in a given segregating population.

Methods presented herein include detecting the presence of one or more marker alleles associated with anthracnose stalk rot resistance in a maize plant and then identifying and/or selecting maize plants that have favorable alleles at those marker loci. Markers listed in Tables 1 and 2 have been identified herein as being associated with the anthracnose stalk rot resistance trait and hence can be used to predict anthracnose stalk rot resistance in a maize plant. Any marker within 50 cM, 40 cM, 30 cM, 20 cM, 15 cM, 10 cM, 9 cM, 8 cM, 7 cM, 6 cM, 5 cM, 4 cM, 3 cM, 2 cM, 1 cM, 0.75 cM, 0.5 cM or 0.25 cM (based on a single meiosis based genetic map) of any of the markers in Tables 1 and 2 could also be used to predict anthracnose stalk rot resistance in a maize plant.

### Marker Assisted Selection

Molecular markers can be used in a variety of plant breeding applications (e.g. see Staub et al. (1996) Hortscience 31: 729-741; Tanksley (1983) Plant Molecular Biology Reporter. 1: 3-8). One of the main areas of interest is to increase the efficiency of backcrossing and introgressing genes using marker-assisted selection (MAS). A molecular marker that demonstrates linkage with a locus affecting a desired phenotypic trait provides a useful tool for the selection of the trait in a plant population. This is particularly true where the phenotype is hard to assay. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by MAS, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite maize line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will allow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The availability of integrated linkage maps of the maize genome containing increasing densities of public maize markers has facilitated maize genetic mapping and MAS. See, e.g. the IBM2 Neighbors maps, which are available online on the MaizeGDB website.

The key components to the implementation of MAS are: (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs, can be used in marker assisted selection protocols.

SSRs can be defined as relatively short runs of tandemly repeated DNA with lengths of 6 bp or less (Tautz (1989) Nucleic Acid Research 17: 6463-6471; Wang et al. (1994) Theoretical and Applied Genetics, 88:1-6) Polymorphisms arise due to variation in the number of repeat units, probably caused by slippage during DNA replication (Levinson and Gutman (1987) Mol Biol Evol 4: 203-221). The variation in repeat length may be detected by designing PCR primers to the conserved non-repetitive flanking regions (Weber and May (1989) Am J Hum Genet. 44:388-396). SSRs are highly suited to mapping and MAS as they are multi-allelic, codominant, reproducible and amenable to high throughput automation (Rafalski et al. (1996) Generating and using DNA markers in plants. In: Non-mammalian genomic analysis: a practical guide. Academic press. pp 75-135).

Various types of SSR markers can be generated, and SSR profiles can be obtained by gel electrophoresis of the amplification products. Scoring of marker genotype is based on the size of the amplified fragment. An SSR service for maize is available to the public on a contractual basis by DNA Landmarks in Saint-Jean-sur-Richelieu, Quebec, Canada.

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002). Plant Mol Biol 48, 539-547; Rafalski (2002b), supra).

SNP markers detect single base pair nucleotide substitutions. Of all the molecular marker types, SNPs are the most abundant, thus having the potential to provide the highest genetic map resolution (Bhattramakki et al. 2002 Plant Molecular Biology 48:539-547). SNPs can be assayed at an even higher level of throughput than SSRs, in a so-called ‘ultra-high-throughput’ fashion, as they do not require large amounts of DNA and automation of the assay may be straight-forward. SNPs also have the promise of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing and coded spheres. Such methods have been reviewed in: Gut (2001) Hum Mutat 17 pp. 475-492; Shi (2001) Clin Chem 47, pp. 164-172; Kwok (2000) Pharmacogenomics 1, pp. 95-100; and Bhattramakki and Rafalski (2001) Discovery and application of single nucleotide polymorphism markers in plants. In: R. J. Henry, Ed, Plant Genotyping: The DNA Fingerprinting of Plants, CABI Publishing, Wallingford. A wide range of commercially available technologies utilize these and other methods to interrogate SNPs including Masscode™ (Qiagen), INVADER® (Third Wave Technologies) and Invader PLUS®, SNAPSHOT® (Applied Biosystems), TAQMAN® (Applied Biosystems) and BEADARRAYS® (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than single SNPs and can be more descriptive of any particular genotype. For example, a single SNP may be allele ‘T’ for a specific line or variety with anthracnose stalk rot resistance, but the allele ‘T’ might also occur in the maize breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms known to those of ordinary skill in the art makes this process highly efficient and effective.

Many of the PHM markers presented herein can readily be used as FLP markers to select for the gene loci on chromosome 10, owing to the presence of insertions/deletion polymorphisms. Primers for the PHM markers can also be used to convert these markers to SNP or other structurally similar or functionally equivalent markers (SSRs, CAPs, indels, etc.), in the same regions. One very productive approach for SNP conversion is described by Rafalski (2002a) Current opinion in plant biology 5 (2): 94-100 and also Rafalski (2002b) Plant Science 162: 329-333. Using PCR, the primers are used to amplify DNA segments from individuals (preferably inbred) that represent the diversity in the population of interest. The PCR products are sequenced directly in one or both directions. The resulting sequences are aligned and polymorphisms are identified. The polymorphisms are not limited to single nucleotide polymorphisms (SNPs), but also include indels, CAPS, SSRs, and VNTRs (variable number of tandem repeats). Specifically with respect to the fine map information described herein, one can readily use the information provided herein to obtain additional polymorphic SNPs (and other markers) within the region amplified by the primers listed in this disclosure. Markers within the described map region can be hybridized to BACs or other genomic libraries, or electronically aligned with genome sequences, to find new sequences in the same approximate location as the described markers.

In addition to SSR's, FLPs and SNPs, as described above, other types of molecular markers are also widely used, including but not limited to expressed sequence tags (ESTs), SSR markers derived from EST sequences, randomly amplified polymorphic DNA (RAPD), and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the maize species, or even across other species that have been genetically or physically aligned with maize, such as rice, wheat, barley or sorghum.

In general, MAS uses polymorphic markers that have been identified as having a significant likelihood of co-segregation with a trait such as the anthracnose stalk rot resistance trait. Such markers are presumed to map near a gene or genes that give the plant its anthracnose stalk rot resistant phenotype, and are considered indicators for the desired trait, or markers. Plants are tested for the presence of a desired allele in the marker, and plants containing a desired genotype at one or more loci are expected to transfer the desired genotype, along with a desired phenotype, to their progeny. Thus, plants with anthracnose stalk rot resistance can be selected for by detecting one or more marker alleles, and in addition, progeny plants derived from those plants can also be selected. Hence, a plant containing a desired genotype in a given chromosomal region (i.e. a genotype associated with anthracnose stalk rot resistance) is obtained and then crossed to another plant. The progeny of such a cross would then be evaluated genotypically using one or more markers and the progeny plants with the same genotype in a given chromosomal region would then be selected as having anthracnose stalk rot resistance.

Markers were identified from linkage mapping as being associated with the anthracnose stalk rot resistance trait. Reference sequences for the markers are represented by SEQ ID NOs:1-16. SNP positions are identified within the marker sequences.

The SNPs could be used alone or in combination (i.e. a SNP haplotype) to select for a favorable QTL allele associated with anthracnose stalk rot resistance. For example, a SNP haplotype at the chromosome 10 QTL disclosed herein can comprise: a “T” at sbd_INBREDA_4, a “C” at sbd_INBREDA_9, a “T” at sbd_INBREDA_13, a “T” at sbd_INBREDA_24, a “T” at sbd_INBREDA_25, a “C” at sbd_INBREDA_32, an “A” at sbd_INBREDA_33, a “G” at sbd_INBREDA_35, an “A” at sbd_INBREDA_093, a “G” at sbd_INBREDA_109, or any combination thereof.

The skilled artisan would expect that there might be additional polymorphic sites at marker loci in and around the chromosome 10 markers identified herein, wherein one or more polymorphic sites is in linkage disequilibrium (LD) with an allele at one or more of the polymorphic sites in the haplotype and thus could be used in a marker assisted selection program to introgress a QTL allele of interest. Two particular alleles at different polymorphic sites are said to be in LD if the presence of the allele at one of the sites tends to predict the presence of the allele at the other site on the same chromosome (Stevens, Mol. Diag. 4:309-17 (1999)). The marker loci can be located within 5 cM, 2 cM, or 1 cM (on a single meiosis based genetic map) of the anthracnose stalk rot resistance trait QTL.

The skilled artisan would understand that allelic frequency (and hence, haplotype frequency) can differ from one germplasm pool to another. Germplasm pools vary due to maturity differences, heterotic groupings, geographical distribution, etc. As a result, SNPs and other polymorphisms may not be informative in some germplasm pools.

### Plant Compositions

Maize plants identified and/or selected by any of the methods described above are also of interest.

### EXAMPLES

The following examples are offered to illustrate, but not to limit, the claimed subject matter. It is understood that the examples and embodiments described herein are for illustrative purposes only, and persons skilled in the art will recognize various reagents or parameters that can be altered without departing from the spirit of the disclosure or the scope of the appended claims.

### Example 1

### Creation of Population with Increased Resistance to Anthracnose Stalk Rot

An F₁-derived DH mapping population for Anthracnose Stalk Rot (ASR) resistance was created from a cross between INBRED A and INBRED B in order to identify QTL that are associated with resistance to ASR. INBRED A is resistant to ASR in contrast to INBRED B. The resulting mapping population displayed varying degrees of resistance.

The F₁DH population was analyzed using ILLUMINA® SNP Genotyping (768 array for the NSS heterotic group). The population was planted in the field in three replicates at one location in Brazil, and phenotyped for ANTROT, ANTINODES, and ANTGR75. The phenotype ANTINODES represents the number of internodes that are infected by the pathogen and includes the internode that was inoculated. Scores for ANTINODES range from 1 to 5 with a 1 corresponding to resistance and a 5 corresponding to susceptibility. The phenotype ANTGR75 represents the number of internodes that are infected at >75%. Scores for ANTGR75 range from 1 to 5 with a 1 corresponding to resistance and a 5 corresponding to susceptibility. ANTSUM is the sum of the ANTINODES and ANTGR75 phenotypes, and the range of ANTSUM is from 1 (Resistant) to 10 (Susceptible).

SNP variation was used to generate specific haplotypes across inbreds at each locus. This data was used for identifying associations between alleles and anthracnose stalk rot resistance at the genome level. Resistance scores and genotypic information were used for QTL interval mapping in MaxQtl and the R package qtl. A QTL for resistance to anthracnose stalk rot was identified on Chromosome 10 between 10-90 cM on a proprietary single meiosis based genetic map

### Example 2

### Determination of Effect of South American QTL for Resistance to Anthracnose Stalk Rot in North America

Progeny from the F₁DH population were sent to a North American breeding station to determine the efficacy of the resistance provided by INBRED A with respect to races of the fungus Colletotrichum graminicola originating in North American. The effect was measured, and the resistant progeny scored 4.5 points better compared to the progeny that were susceptible. The per se score of the parents used to create this F₁DH population were 1.52 and 9.88 for INBRED A and INBRED B, respectively.

The effect was measured in North America by crossing F₁DH lines to a tester, phenotyped in 2011, crossed with a tester, INBRED E, to determine the effect of the resistance in a hybrid. While not as strong as the effect seen in the inbreds per se, a 1.5 point score improvement of the F₁DH/TC lines with the chromosome 10 region from INBRED A was observed.

### Example 3

### Initial Population Development for Fine-Mapping

BC₂-derived populations were developed in several susceptible backgrounds, including inbred PH1M6A (U.S. Pat. No. 8,884,128) and PH1KYM (U.S. Pat. No. 8,692,093), i.e. they were used as recurrent parents. Different sections of the resistance locus between 10 and 90 cM (on the PHB map, a proprietary single meiosis based genetic map) from INBRED A were selected for by marker assisted selection. Individual plants of the BC₂ progeny from these populations were inoculated with Colletotrichum graminicola and phenotyped. Genotypic data was generated using TAQMAN® markers selected for heterozygosity between parents of the respective crosses in the region of interest on Chromosome 10. The phenotypes, ANTINODES and ANTGR75, were used to assess response to infection with Colletotrichum graminicola, and the genotypes were analyzed with the TIBCO SPOTFIRE® data analysis and visualization tool, which employs a Kruskal-Wallis methodology to determine the p-value and defines the association between phenotype and genotype. A p-value of 1.00E-030 was obtained for marker C002DC9-001 located on C10 at 32.9 cM on the proprietary single meiosis-based genetic map, representing a strong association between genotype and phenotype. The QTL region was further refined to a region of chromosome 10 from 13.3-39.7 cM (single meiosis based genetic map).

To further refine the region of interest on C10, 24 markers from the ILLUMINA® SNP Genotyping 50 k-plex assay, that were identified to be polymorphic between the resistant donor line INBRED A and the susceptible recurrent parent lines, PH1M6A and INBRED D, were converted to KASPar markers (method is known to 15 one of ordinary skill in the art). Testing of the parents of the population and subsequent testing of a small panel of recombinant BC₂ lines within the 10-40 cM region identified four markers that further refined the QTL area to 18-40 cM (i.e. the region was delimited by markers C00429-801 and PHM824-17). The markers used for genotyping and the p-values of the marker-trait associations are displayed in Table 1.

TABLE 1
Markers having the most significant association with the phenotype in each of two populations

| | | | IBM2 | | | P-value | P-value | Marker | |
|---|---|---|---|---|---|---|---|---|---|
| | | B73 physical | genetic | NBRED | PH1M6A& | (PH1M6A = | PH1KYM = | Reference | SNP |
| Marker | PHB | map position | map | A | PH1KYM | recurrent) | recurrent) | Sequence | POSITION |
| C00429-801 | 18 | 2234232 | 6.47 | T | A | 2.02E−27 | 8.06E−12 | SEQ ID | 84 |
| | | | | | | | | NO: 1 | |
| SYN17615 | 17.88 | 2437860 | 6.85 | C | A | 3.14E−29 | 1.11E−11 | SEQ ID | 61 |
| | | | | | | | | NO: 2 | |
| PZE-110006361 | 32.9 | 4899391 | 19.55 | G | T | 5.55E−30 | 3.29E−21 | SEQ ID | 51 |
| | | | | | | | | NO: 3 | |
| PHM824-17 | 39.7 | 5646609 | 23.8 | C | T | 2.35E−20 | 2.92E−15 | SEQ ID | 278 |
| | | | | | | | | NO: 4 | |

### Example 4

### Further Population Development for Fine-Mapping and Evaluation of the INBRED A Region in Different Elite Line Backgrounds

BC₃S₂ populations were generated using 8 susceptible North American elite lines as recurrent parents. Different sections of the resistant locus from INBRED A, with emphasis on the region between 18 and 44 cM (PHI map), were selected for by marker assisted selection.

Two of the BC₃S₂ populations, with PH1M6A and PH17JT (U.S. Pat. No. 8,481,823) as the recurrent parents, were used to fine-map the QTL region further. Approximately 1300 progeny plants from both populations were planted in the field, inoculated with C. graminicola, and phenotyped for ANTINODES and ANTGR75.

To determine if the QTL region derived from INBRED A had a consistent effect across a panel of different susceptible genetic backgrounds, BC₃S₂ lines from each of the above mentioned recurrent parents, that were either homozygous for the INBRED A donor or the recurrent parent in the chromosome 10 region of interest, were planted as single rows, with two replications. The plants were inoculated and phenotyped for ANTINODES and ANTGR75.

Markers in the chromosome 10 region from 17-44 cM on the internally derived single meiosis based genetic map were used to genotype both the large segregating BC₃S₂ populations and the fixed BC₃S₂ lines with the different recurrent parent backgrounds.

For the large mapping populations, associations between phenotypes and genotypes were analyzed using the TIBCO SPOTFIRE® data analysis and visualization tool. Two markers C01964-1 and C01957-1 were identified as showing a strong association with ANTINODES and ANTGR75. (P-values of 1.33E-62 and 4.80E-62, respectively)

For the population with PH1 M6A as the recurrent parent, the average ANTSUM score for individuals with the INBRED A allele was 2.6. Heterozygotes had a score of 3.0 and individuals with the PH1 M6A haplotype had a score of 6.2. For the population with PH17JT as the recurrent parent, the average ANTSUM score for individuals with the INBRED A allele was 3.4. Heterozygotes had a score of 3.5 and individuals with the PH17JT haplotype had a score of 5.3.

The fact that the heterozygote individuals have a similar level of resistance than the individuals homozygous for the INBRED A allele, indicates that the INBRED A-derived QTL has a dominant effect. An ANTSUM score improvement of 1.9 (PH17JT background) to 3.6 (PH1M6A) points is a major effect.

The number of fixed BC₃S₂ Near Isogenic Lines (NILs) for the eight different recurrent parent backgrounds ranged from 4 to 23 lines per background. The improvement in ANTSUM score for the NILs with the INBRED A background versus the NILs with the recurrent parent background ranged from a 1.1 score difference to a 3.9 score difference, depending on the recurrent parent background.

### Example 5

### Additional Marker Development

Exome capture sequence data derived from four pairs of INBRED A×recurrent parent NIL-bulks (Recurrent parents: PH1M1Y (U.S. Pat. No. 8,604,313), INBRED C, INBRED D, and PH17JT) was utilized to identify additional polymorphic SNPs in the C10:18-40 cM region. For each recurrent parent background there is a “bulk with” and a “bulk without” the region of interest. SNPs that were polymorphic in the chromosome 10 region of interest between the INBRED A positive bulk and all four of the recurrent parent bulks were identified. A subset of SNPs was chosen to develop KASPar markers using the SNP flanking sequence to develop primers. The KASPar markers were assayed against INBRED A and the recurrent parents. Markers that were diagnostic between parents were then screened against recombinants from the BC₃S₂ population, PH1M6A<4[INBRED A]. With these additional markers (See Table 2) the region encompasses a 1 Mb region flanked by SYN17244 and sbd_INBREDA_48. The INBRED A marker alleles in Table 2, as well as marker alleles in linkage disequilibrium with the INBRED A marker alleles in Table 2, can be used to identify and select maize plants with increased anthracnose stalk rot resistance. Additional KASPAR markers were developed, further delimiting the region to an interval defined by and including sbd_INBREDA_093 and sbd_INBREDA_109. The association between the trait and marker sbd_INBREDA_093 had a p-value of 1.93 E-051, while the association between the trait and marker sbd_INBREDA_109 had a p-value of 7.82 E-049.

TABLE 2
Marker alleles for marker assisted selection

| | | | | | SNP |
|---|---|---|---|---|---|
| | | IFavorable | Unfa- | | Position |
| | | allele | vor- | Marker | in |
| | | (INBRED | able | Reference | Reference |
| Marker | PHB | A) | allele | Sequence | Sequence |
| SYN17244 | 25.1 | T | C | SEQ ID | 61 |
| | | | | NO: 5 | |
| sbd_INBREDA_093 | N/A | A | G | SEQ ID | 51 |
| | | | | NO: 15 | |
| sbd_INBREDA_4 | 25.7 | T | A | SEQ ID | 51 |
| | | | | NO: 6 | |
| sbd_INBREDA_9 | 26.11 | C | G | SEQ ID | 51 |
| | | | | NO: 7 | |
| sbd_INBREDA_13 | 26.18 | T | A | SEQ ID | 51 |
| | | | | NO: 8 | |
| sbd_INBREDA_24 | 26.49 | T | A | SEQ ID | 51 |
| | | | | NO: 9 | |
| sbd_INBREDA_25 | 26.49 | T | G | SEQ ID | 51 |
| | | | | NO: 10 | |
| sbd_INBREDA_32 | 27.52 | C | T | SEQ ID | 51 |
| | | | | NO: 11 | |
| sbd_INBREDA_33 | 27.52 | A | C | SEQ ID | 51 |
| | | | | NO: 12 | |
| sbd_INBREDA_35 | 27.52 | G | A | SEQ ID | 51 |
| | | | | NO: 13 | |
| sbd_INBREDA_109 | N/A | G | A | SEQ ID | 51 |
| | | | | NO: 16 | |
| sbd_INBREDA_48 | 28.52 | T | C | SEQ ID | 51 |
| | | | | NO: 14 | |

### Example 6

### Effect of Introgression of Inbred a Region

The Inbred A region was introgressed into mid-maturity maize (North American) lines as described in Example 5. The resulting plants were then testcrossed to an inbred tester line, and the hybrids were phenotyped. Table 3 shows the average ANTSUM effects for the different backgrounds. The presence of the Inbred A region resulted in an increase in resistance in all cases.

TABLE 3
Average ANTSUM effects in different backgrounds

| | | ANTSUM score |
|---|---|---|
| Tester: INBRED E | | |
| PH1M1Y < 4[INBRED A] | +region | 1.3 |
| PH1M1Y < 4[INBRED A] | −region | 5.3 |
| INBRED C < 4[INBRED A] | +region | 1.8 |
| INBRED C < 4[INBRED A] | −region | 6.1 |
| PH1D84 < 4[INBRED A] | +region | 1.9 |
| PH1D84 < 4[INBRED A] | −region | 4.3 |
| Tester: INBRED F | | |
| PH1M6A < 4[INBRED A] | +region | 2.6 |
| PH1M6A < 4[INBRED A] | −region | 6.4 |
| PH1V5T < 4[INBRED A] | +region | 2.6 |
| PH1V5T < 4[INBRED A] | −region | 4.1 |
| PH17JT < 4[INBRED A] | +region | 2.6 |
| PH17JT < 4[INBRED A] | −region | 5.4 |
| PH1KYM < 4[INBRED A] | +region | 2.9 |
| PH1KYM < 4[INBRED A] | −region | 6.1 |

*PH18D4 is disclosed in U.S. Pat. No. 8,759,636
*PH1V5T is disclosed in U.S. Pat. No. 8,907,160

What is claimed:

1. A method of identifying and/or selecting a maize plant with anthracnose stalk rot resistance, said method comprising:
a. analyzing DNA of a maize plant for the presence of a QTL allele on chromosome 10 that is associated with anthracnose stalk rot resistance, wherein said QTL allele comprises:
i. a “T” at sbd_INBREDA_4,
ii. a “C” at sbd_INBREDA_9,
iii. a “T” at sbd_INBREDA_13,
iv. a “T” at sbd_INBREDA_24,
v. a “T” at sbd_INBREDA_25,
vi. a “C” at sbd_INBREDA_32,
vii. an “A” at sbd_INBREDA_33, and
viii. a “G” at sbd_INBREDA_35;
b. selecting said maize plant as having anthracnose stalk rot resistance if said QTL allele is detected;
c. crossing the maize plant selected in (b) with a second maize plant; and
d. obtaining a progeny plant that has the QTL allele.

2. The method of claim 1, wherein said QTL allele is located in a chromosomal interval defined by and including markers C00429-801 and PHM824.

3. The method of claim 1, wherein said QTL allele is located in a chromosomal interval defined by and including markers SYN17244 and sbd_INBREDA_48.

4. The method of claim 1, wherein said QTL allele is located in a chromosomal interval defined by and including markers sbd_INBREDA_093 and sbd_INBREDA_109.

5. The method of claim 1, wherein said analyzing comprises isolating nucleic acids and detecting one or more marker alleles linked to and associated with said QTL allele.

6. A method of identifying and/or selecting a maize plant with anthracnose stalk rot resistance, said method comprising:
a. detecting in a maize plant at least one marker allele that is linked to and associated with one or more marker alleles selected from the group consisting of:
i. a “T” at sbd_INBREDA_4,
ii. a “C” at sbd_INBREDA_9,
iii. a “T” at sbd_INBREDA_13,
iv. a “T” at sbd_INBREDA_24,
v. a “T” at sbd_INBREDA_25,
vi. a “C” at sbd_INBREDA_32,
vii. an “A” at sbd_INBREDA_33, and
viii. a “G” at sbd_INBREDA_35;
b. selecting said maize plant that has the at least one marker allele that is linked to and associated with one or more marker alleles set forth in (i)-(viii) of step (a);
c. crossing the maize plant selected in (b) with a second maize plant; and
d. obtaining a progeny plant that has the at least one marker allele that is linked to and associated with one or more marker alleles set forth in (i)-(viii) of step (a).

7. The method of claim 6, wherein the at least one marker allele is linked to any of (i)-(viii) of step (a) by 10 cM on a single meiosis based genetic map.

8. The method of claim 6, wherein the at least one marker allele is linked to any of (i)-(viii) of step (a) by 2 cM on a single meiosis based genetic map.

9. A method of identifying and/or selecting a maize plant with anthracnose stalk rot resistance, said method comprising:
a. detecting in a maize plant at least one marker allele that is linked to and associated with a haplotype comprising:
i. a “T” at sbd_INBREDA_4,
ii. a “C” at sbd_INBREDA_9,
iii. a “T” at sbd_INBREDA_13,
iv. a “T” at sbd_INBREDA_24,
v. a “T” at sbd_INBREDA_25,
vi. a “C” at sbd_INBREDA_32,
vii. an “A” at sbd_INBREDA_33, and
viii. a “G” at sbd_INBREDA_35;
b. selecting said maize plant that has the at least one marker allele that is linked to and associated with the haplotype;
c. crossing the maize plant selected in (b) with a second maize plant; and
d. obtaining a progeny plant that has at least one marker allele that is linked to and associated with the haplotype in (a).

10. The method of claim 9, wherein the at least one marker allele is linked to the haplotype by 10 cM on a single meiosis based genetic map.

11. The method of claim 9, wherein the at least one marker allele is linked to the haplotype by 2 cM on a single meiosis based genetic map.

12. A method of identifying and/or selecting a maize plant with anthracnose stalk rot resistance, said method comprising:
a. detecting in the maize plant a haplotype comprising:
i. a “T” at sbd_INBREDA_4,
ii. a “C” at sbd_INBREDA_9,
iii. a “T” at sbd_INBREDA_13,
iv. a “T” at sbd_INBREDA_24,
v. a “T” at sbd_INBREDA_25,
vi. a “C” at sbd_INBREDA_32,
vii. an “A” at sbd_INBREDA_33, and
v. a “G” at sbd_INBREDA_35;
b. selecting said maize plant that has said haplotype;
c. crossing the maize plant selected in (b) with a second maize plant; and
d. obtaining a progeny plant that has the haplotype in (a).

13. A method of introgressing a QTL allele associated with anthracnose stalk rot resistance into a maize plant said method comprising:
a. screening a population with at least one marker to determine if one or more maize plants from the population comprises a QTL allele associated with anthracnose stalk rot resistance, wherein the QTL allele comprises:
i. a “T” at sbd_INBREDA_4,
ii. a “C” at sbd_INBREDA_9,
iii. a “T” at sbd_INBREDA_13,
iv. a “T” at sbd_INBREDA_24,
v. a “T” at sbd_INBREDA_25,
vi. a “C” at sbd_INBREDA_32,
vii. an “A” at sbd_INBREDA_33, and
viii. a “G” at sbd_INBREDA_35;
b. selecting from said population at least one maize plant comprising the QTL allele;
c. crossing the maize plant selected in (b) with a second maize plant; and
d. obtaining a progeny plant that comprises the QTL allele.

14. The method of claim 13, wherein the at least one marker used for screening is located within 5 cM on a single meiosis based genetic map of any one of (i)-(viii) of step (a).

15. The method of claim 13, wherein the at least one marker used for screening is located within 1 cM on a single meiosis based genetic map of any one of (i)-(viii) of step (a).


SEQUENCE LISTING (16 sequences)

SEQ ID NO: 1 - DNA, 201 bp, artificial
  C00429-801 Reference sequence
tcaggtatatgattcagccaagttggcaaccaggtgttagtcgggctgantgattcaaatnccatgtggaatcaagaccactgwttcaggcaattctataatgcaactttgaattgatttcgctgtttttaccaaaactctgaagaaattntgancggctcaggtgatcaggaaacagacagtaaaaatttcatagatcta

SEQ ID NO: 2 - DNA, 121 bp, artificial
  SYN17615 Reference sequence
ctagctgctttactaaatgtgcagccttaacatcaatggttgggagatgatgatctccatmggcttcctggctgcaacagggtacgtacgcaaccacatcagtttcttatctttttttaat

SEQ ID NO: 3 - DNA, 101 bp, artificial
  PZE-110006361 Reference sequence
tgaaagtttagttttgaaatgctgtaaccgaatagagcggcaaagaatatkgggaaggctgctagaactatagctgacagtggtagcaattcacgtttgaa

SEQ ID NO: 4 - DNA, 354 bp, artificial
  PHM824-17 Reference sequence
acgtcttcttctccgaggnccagttcatcccggccgaggacctcgccgccatcgacgggctctggaaggagcacagcggcggcaggttcgggtacagcgtgcagcggcggctctgggagaagtcgcggcgcgacttcacccgcttcttcatccgggtcggctggatgaggaagctggacacggaggtggagcagtacaactacagggccttccccgacgagttcntgtggganttgacggacgacacgcccgagggacacctgccgctcaccaacgcyctcaggggcacgcagctcctggcgaacatcctcacccacccggccttccaggaggaggaccagngagacggagctg

SEQ ID NO: 5 - DNA, 121 bp, artificial
  SYN17244 Reference sequence
tagcagcagcgacancgcggaggtgcacatgtcagcctcaagaccgggatccgctgctgcygcgtcgtcctcgtcctcctctctcagtctcagctgcaacaagcacaacccgcaggccgcc

SEQ ID NO: 6 - DNA, 102 bp, artificial
  sbd_INBREDA_4 Reference sequence
gatgcagcatgtggtgacacccaagaggaggagggcatccataagttaatwatcatgacgctgtcgtgtaattttaggaggataccgtgtgtgcatggcttg

SEQ ID NO: 7 - DNA, 102 bp, artificial
  sbd_INBREDA_9 Reference sequence
cttgtcgagggtgtagcagagcactacagaaccgaggaaggcgaagcagascatccatctcctctgatgagttgtgcgaccaagttcaccttggaaccagtt

SEQ ID NO: 8 - DNA, 102 bp, artificial
  sbd_INBREDA_13 Reference sequence
agaatttcgggcggggagtcaggtttcagatcaccattgccatatctgtcwaagctattttgactgggtaggctatcattgacatccatctgttgctgcaat

SEQ ID NO: 9 - DNA, 102 bp, artificial
  sbd_INBREDA_24 Reference sequence
caaagggggaaatagcagtagagactggagattggtgatcttacacaagtwaagtaaatcatgggcgcatggagctggaatcgacaacaagctcgaggccgc

SEQ ID NO: 10 - DNA, 102 bp, artificial
  sbd_INBREDA_25 Reference sequence
gggaacgcaggattgctgacgatagaatatctgttttttgttgtggaggakaccggtggtgctgctgactgctgactgctcgctgggagaggaaagcagcgt

SEQ ID NO: 11 - DNA, 102 bp, artificial
  sbd_INBREDA_32 Reference sequence
agttgccagtgaagtgaggccttcaaggcaaatagctaaagcttcatctayaatattgcatgacgaaagggaaagtttacaaagtcctaatggtagaaccac

SEQ ID NO: 12 - DNA, 102 bp, artificial
  sbd_INBREDA_33 Reference sequence
ggtaagccttctagcaaactgcaattattaagctcaaaacgttccaaattmttaaaataggatcgctcaagtaaccacccaggatatgtgtcagatttgtaa

SEQ ID NO: 13 - DNA, 102 bp, artificial
  sbd_INBREDA_35 Reference sequence
tttcaaatgaacgacttgttcacagcaaacagcggccggaagtgtctcacrtcgagtagtcaccaaaaccttgctccccgactgtttagagactaatggagc

SEQ ID NO: 14 - DNA, 102 bp, artificial
  sbd_INBREDA_48 Reference sequence
gctttgtaacagcactgcaaaaaatctgtgcgtccctttgacgctgatgcygaccttttagaaatgaaggctctatcacaagctcttggcattcccttgcac

SEQ ID NO: 15 - DNA, 102 bp, artificial
  sbd_INBREDA_093 reference sequence
gctgcggtcagagctggcacggatgcgggcctacctgtcagggatggagcrcagcaaagggggccggtcaacgccgccgtcctcgccgtctcggagggcaaa

SEQ ID NO: 16 - DNA, 102 bp, artificial
  sbd_INBREDA_109 reference sequence
catttgcaagatccgtcaagacgtcgaagtggccgaaactgccgctagcartgtcctacggctagacccagacgacgcgtcggtttacattcttctctctaa